(19) United States
(12) Patent Application Publication
(10) Pub. No.: US 2018/0008802 A1
(43) Pub. Date: Jan. 11, 2018

(54) MEDICAL APPARATUS

(71) Applicants: Ramesh Ganapathy, Surrey (GB); Alok S. Godse, Newcastle Upon Tyne (GB)

(72) Inventors: Ramesh Ganapathy, Surrey (GB); Alok S. Godse, Newcastle Upon Tyne (GB)

(21) Appl. No.: 15/643,560

(22) Filed: Jul. 7, 2017

(30) Foreign Application Priority Data
Jul. 11, 2016 (GB) GB1611994.3

(51) Int. Cl. A61M 25/00; A61M 25/01; A61M 25/06

(52) U.S. Cl. CPC A61M 25/0071; A61M 25/065; A61M 25/0113; A61M 25/007; A61M 25/0074; A61M 25/0108

(57) ABSTRACT

A medical catheter that includes an elongate main tube, the tube having an outer wall that surrounds an internal passage, branch tubes having an outer wall connected to the outer wall of the main tube which extend outward from the main tube in different respective directions and have one or more apertures passing through their outer wall to their internal passage to permit fluid to pass through the branch tubes and into the main tube in use. The branch tubes are arranged to be able to move towards each other to permit location of the catheter within the bore of a needle or needle-like apparatus and which return to their original outwards state when removed.

### CROSS-REFERENCE TO RELATED APPLICATIONS AND PRIORITY

The present application claims priority from UK Patent Application No. GB1611994.3 filed on Jul. 11, 2016, the entirety of which is hereby incorporated by reference.

### FIELD OF THE INVENTION

This invention relates to a medical apparatus, particularly a catheter apparatus, and also to a medical instrument apparatus for locating the catheter in place.

### BACKGROUND OF THE INVENTION

Catheters are commonly used in medical procedures. A catheter is typically a tube formed of medical-grade plastic(s) material that is inserted into a patient's body to provide various functions.

For example, catheters may be used to drain fluid or air trapped within a body cavity, or to deliver fluids or drugs to the body. Some catheters are left inside the body, e.g. to act as shunts whereby fluid from one part of the body is drained to another part of the body.

Inserting a catheter is a relatively complicated process depending on the type of procedure. For example, in a chest drain procedure, an incision is first made to the skin. A needle is then inserted into the pleural cavity and aspiration is performed using a syringe to confirm correct placement. The syringe is then removed from the needle and a guide wire threaded through the needle and into the cavity. The needle is then removed leaving the wire in place and a dilator is passed over the wire to widen the entry hole. The dilator is then removed and the catheter with connected trocar is passed over the wire and into position extending between the pleural cavity and the exterior so that drainage can take place. The catheter is usually sutured to the outer skin and a bag or other instrument connected to the catheter. The process typically takes 10-15 minutes.

Sometimes, the catheter can become displaced from its location within the body. For this purpose, some catheters comprise an inflatable balloon part which is inflated after insertion to retain it in position. This makes the catheter rather complex in terms of structure, and requires appropriate tubing and an inflation control device.

### SUMMARY OF THE INVENTION

A first aspect of the invention provides a medical catheter, including an elongate main tube, the tube having an outer wall that surrounds an internal passage; first and second branch tubes, each having an outer wall connected to the outer wall of the main tube, in which the outer wall surrounds an internal passage that is in communication with that of the main tube, wherein the branch tubes extend outwards from the main tube in different respective directions and have one or more apertures passing through their outer wall to their internal passage to permit fluid to pass through the branch tubes and into the main tube in use, and wherein the branch tubes are arranged so as to be resiliently movable, generally towards each other, to permit location of the catheter within the bore of a needle or needle-like apparatus and which return to their original outwards state when removed.

At least part of the main tube may extend along a central axis and in which the branch tubes extend outwardly from said central axis on opposite sides to provide an overall Y-shape or similar. The branch tubes may extend from one end of the main tube.

Each branch tube may include a plurality of apertures passing through the side wall. The apertures may be distributed along the branch tube, between the connection with the main tube and a free end of the branch tube.

The first and second branch arms may be connected at or near one end of the main tube, and at or near the opposite end may be provided third and fourth branch arms which extend away from the main tube in different respective directions and have one or more apertures passing through their outer wall to their internal passage to permit fluid to pass through the branch tubes and into the main tube in use, and wherein the third and fourth branch tubes are arranged so as to be resiliently movable, generally towards each other.

One or more apertures may be further provided in the wall of the main tube.

The catheter may further comprise a radiopaque marker. The radiopaque marker may extend along the main tube. One or more markers may be provided on respective branch tube(s). A marker may be provided on or near the free end of each branch tube.

A second aspect of the invention provides a medical catheter comprising a main tube having first and second ends, and at least two, shorter, branch tubes which extend outwardly from at, or near, the first end of the main tube on opposite sides to form an approximate Y-shape, the branch tubes each having one or more apertures formed through their side wall so as to provide a passage from the branch tube into the main tube, the branch tubes being connected to the main tube in a flexible manner such that they can be moved together and located within a needle or needle-like structure and which are biased so that they return to their original outwards position when removed therefrom.

A plurality of further such branch tubes may be provided at, or near, the second end of the main tube so that the catheter forms an approximate X-shape.

A third aspect provides an apparatus for locating a catheter in a medical procedure, the apparatus including
a barrel having a first end and a second end and a sidewall defining an interior chamber;
a needle or needle-like member having first and second ends and a longitudinal passage extending between said ends, the first end of the needle being connected in use to the second end of the barrel, and the second end of the needle being a free-end, the longitudinal passage being arranged to house a catheter according to any preceding claim;
a plunger locatable within the interior chamber of the barrel for reciprocal movement therein between a non-depressed position and a depressed position and connected to a plunger rod which passes through the barrel second end and part-way along the passage of the needle so as to cause ejection of only part of the catheter from the needle free-end as the plunger is moved towards the depressed position in use.

The apparatus may further comprise a catheter according to any preceding definition, pre-installed within the needle.

The length of the plunger rod may be arranged to cause ejection of part of the catheter sufficient to allow the branch arms to return to the outwards state.

The apparatus may further comprise means provided within the needle passage or the barrel for limiting the travel of the plunger rod to cause ejection of part of the catheter to allow the branch arms to return to the outwards state. The limiting means may comprise at least one indentation within the needle passage.

The apparatus may further comprise a secondary syringe connected to the needle by means of a branch tube for use in aspiration.

There may also be provided a medical kit, comprising an apparatus according to any preceding definition, and one or more of a fluid-collecting bag for connection to the free end of the main tube of a catheter, a scalpel and a retaining member for locating over the catheter for securing it to a patient.

The scalpel may comprise a blade which is marked so as to indicate visually a depth of cut required for a particular medical procedure.

The retaining member may comprise a central arcuate portion arranged to locate around part of the catheter wall, and first and second flaps located on opposite sides of the arcuate portion.

### BRIEF DESCRIPTION OF THE DRAWINGS

The invention will now be described, by way of non-limiting example, with reference to the accompanying drawings, in which:

FIG. 1a is a plan view of a first embodiment catheter in accordance with the invention;

FIG. 1b is a cross sectional view of one part of the 5 catheter shown in FIG. 1a;

FIG. 1c is a cross sectional view of another part of the catheter shown in FIG. 1a;

FIG. 2 is a plan view of the FIG. 1a catheter when positioned within a needle or trocar bore;

FIG. 3a is a side view of a first medical instrument in which the FIG. 1a catheter is pre-installed;

FIG. 3b is a close-up view of part of the first medical instrument prior to use;

FIG. 3c is a close-up view of part of the first medical instrument during use;

FIG. 4 is a sectional view of the FIG. 3a instrument when used in a first medical procedure;

FIG. 5 is a sectional view showing the FIG. 1a catheter in place following withdrawal of the FIG. 3a instrument;

FIG. 6 is a partial side view of a medical scalpel, which may form part of a kit including the FIG. 3a instrument;

FIG. 7a is a plan view of the FIG. 1a catheter with a flap apparatus for securing the catheter to a patient's skin;

FIG. 7b is a side view of the FIG. 7a flap apparatus;

FIG. 8 is a side view of a second medical instrument in which the FIG. 1a catheter is pre-installed;

FIG. 9a is a plan view of a second embodiment catheter in accordance with the invention;

FIG. 9b is a plan view of the FIG. 9a catheter when positioned within a needle or trocar bore;

FIG. 10 is a close-up view of part of a further medical instrument prior to use;

FIG. 11a is a sectional view of the further medical instrument when placing the FIG. 9a catheter in a further medical procedure;

FIG. 11b is a sectional view of the FIG. 9a catheter in place following withdrawal of the instrument;

FIG. 12a a close-up view of a third embodiment catheter in place within a needle or trocar of a medical instrument; and

FIG. 12b is a side view of a medical instrument with the third embodiment catheter pre-installed, shown during a further medical procedure.

### DETAILED DESCRIPTION OF PREFERRED EMBODIMENTS

Embodiments herein provide a medical apparatus for use in medical interventions.

One apparatus is a catheter, being a tube inserted into the body, usually through an incision. A catheter is used typically to drain fluid from a body cavity but in some cases is used to deliver fluid.

Also described is a medical instrument comprised of a syringe or syringe-type apparatus having a hollow needle or trocar for insertion into a patient's body, with a catheter pre-installed within the needle or trocar.

In some situations, a catheter may be referred to as a cannula.

As will become evident, advantages of the catheter and instrument include ease of insertion, including single-handed use. Other advantages include self-retention of the catheter once inserted into the body.

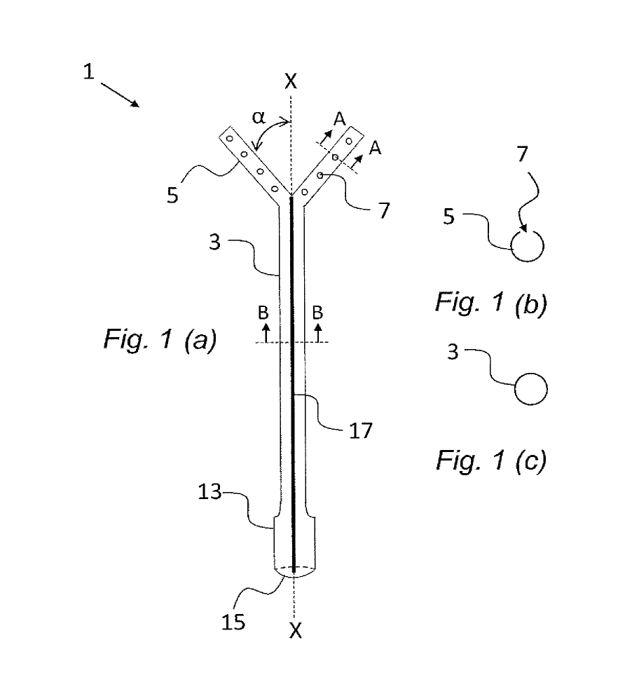

Referring to FIG. 1a, a catheter 1 in accordance with a first embodiment is shown.

The catheter 1 comprises a main, elongate tube 3 extending along lengthwise axis X-X. First and second branch tubes 5 are joined to the proximal end of the main tube 3 such that the internal passages of the tubes are in communication. The branch tubes 5 each extend away from axis X-X in opposite directions at an angle α which in this case is approximately 40 degrees. The catheter 1 therefore has an overall “Y shape” when unrestricted by an external tube or container. Towards the distal end of the catheter 1 is a connector region 13 having an open end 15 for attachment to another tube or bag, e.g. for collecting drained fluids.

The walls of the branch tubes 5 have a plurality of holes 7 passing from the exterior to the interior passage, the holes extending in a line along the longitudinal extent of each branch tube. In some embodiments, holes may be provided on the opposite (rear) side of the branch tubes 5 also. In the shown example, each branch tube 5 has four holes, but different numbers can be used.

The diameter of the tube(s) will vary depending on size and weight of the patient they are to be used with. The holes 7 may be approximately 1 mm in diameter, and may be spaced approximately 2 mm apart.

In some embodiments, a radiopaque marker 17 is provided on the catheter 1. In this case, the marker 17 runs along the lengthwise axis X-X of the first tube 3.

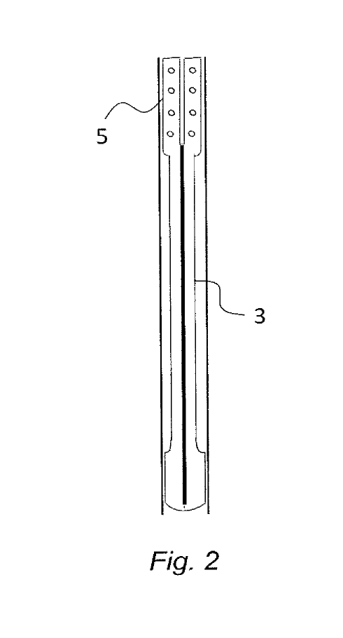

The arrangement of the branch tubes 5 relative to the main tube 3 is such that in use the catheter 1 self-retains within a body cavity once inserted. The catheter 1 also allows fluids to be drained from the cavity through the holes 7. The branch tubes 5 are biased to the shown outwards position by virtue of their moulded shape, and are formed of a suitable medical grade plastics material with resilient characteristics. This is to ensure that when deformed or moved to a different shape, e.g. when the catheter 1 is located within a narrow tube so that the branch tubes 5 are generally parallel with the main tube 3, for which see FIG. 2, they will return to the original shape when removed. In other words, the branch tubes 5 spring-back from the FIG. 2 state to the state shown in FIG. 1a.

The angle α can be any suitable angle, for example from between 40 degrees and 60 degrees. The length of the branch tubes 5 may be, but not essentially, 15 mm.

FIGS. 1b and 1c show sectional views of the catheter 1 for completeness.

The catheter 1 is arranged in use to be introduced to a body cavity as part of a medical procedure. Introduction is by means of an instrument and method to be described below. For illustration, we will now describe its use in draining fluid from a human chest, although it can be used in other procedures.

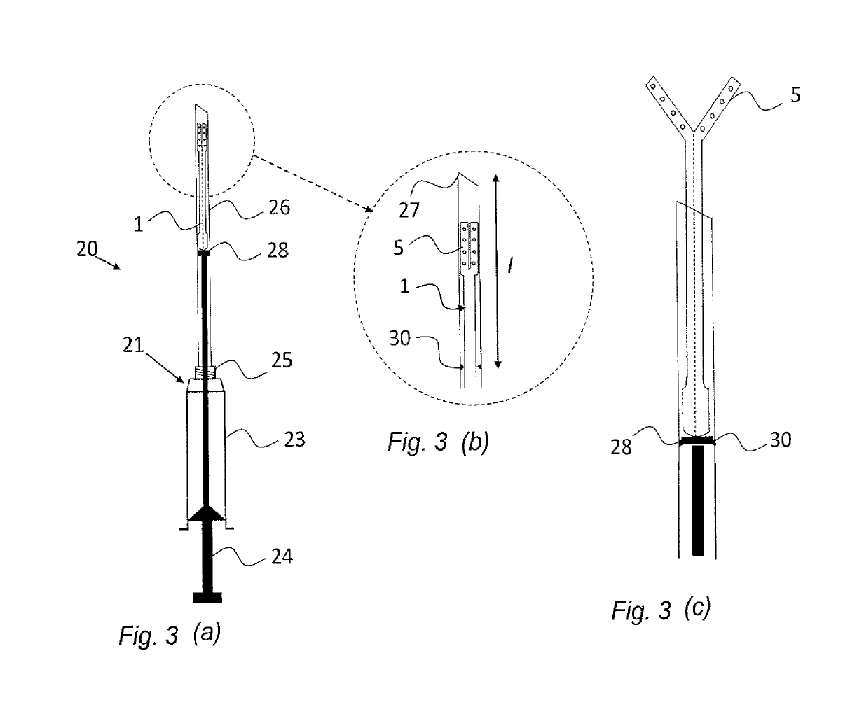

Referring now to FIGS. 3a and 3b, a first example medical instrument 20 is shown for use in a chest draining procedure.

The instrument 20 comprises a syringe, or syringe-type instrument, indicated generally by reference numeral 21, which is largely conventional in structure, having a barrel 23, plunger 24, hub 25 and needle 26. The plunger 24 is connected to a plunger rod which passes through the lower end of the barrel 23 and into the needle 26. Reference numeral 28 indicates the plunger tip at the end of the plunger rod, which is within the bore of the needle 26.

The above-mentioned catheter 1 is provided within the bore of the needle 26, between its open tip 27 and the plunger tip 28 which is located behind. The instrument 20 may be provided with the catheter 1 already installed and contained as shown in its narrowed or collapsed state. The instrument 20 may be provided in a medical kit with other related instruments including one or more of a drainage bag, scalpel and/or instructions for use.

Pressure on the plunger 24 causes the distal end of the catheter 1 to be ejected towards, and out of, the needle tip 27; as the catheter emerges from the tip, the branch tubes 5 spring open to the state shown in FIG. 3c which retains the catheter from returning backwards, e.g. out of a body cavity.

Travel of the plunger tip 28 along the lengthwise extent of the needle bore may be limited by a narrowing region or similar mechanical structure within the bore, or alternatively within the barrel 23. For example, one or more indentations or ridges 30 (see FIG. 3b) may be provided, the lengthwise position of which is arranged to prevent the entire catheter 1 from being ejected from the needle 26 due to pressure on the plunger 24. It should also be positioned appropriately for the proximal end of the catheter to locate outside of the body, in this case. In the shown example, the distance l of the ridges 30 from the needle tip 27 should be appreciably less than the length of the catheter 1 so that the distal end of said catheter will remain external of the body.

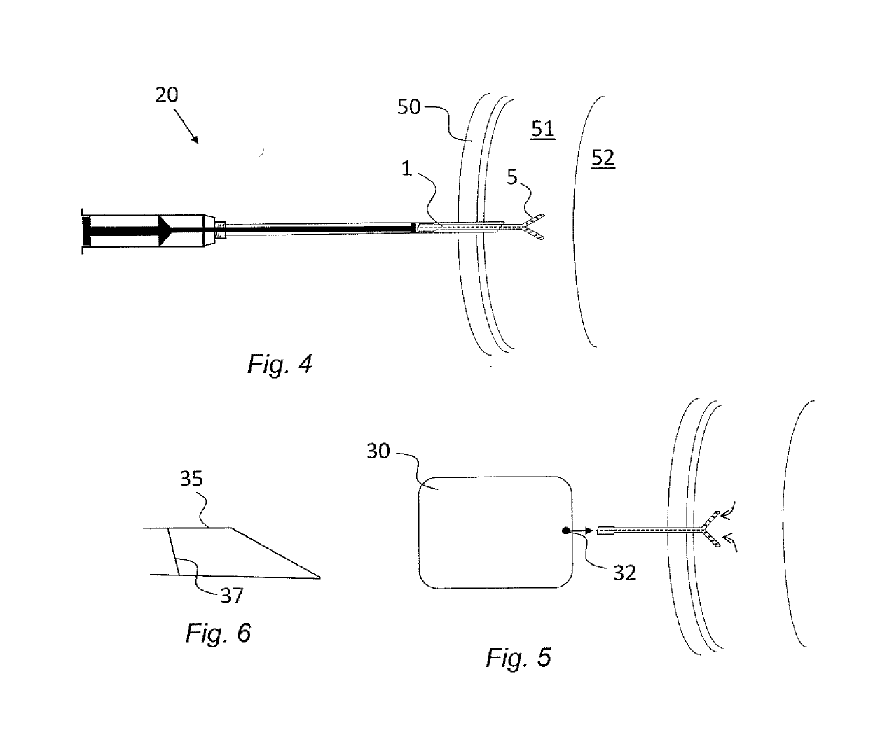

Referring to FIG. 4, the instrument 20 is shown in relation a sectional view of a human chest, comprised of a chest wall 50, pleural space 51 and lung 52. Initially, an incision is made in the skin with a scalpel, and the needle 26 (which in some procedures can be called an introducer or trocar) is introduced so that the tip 27 enters the pleural space 51 containing the fluid. This can be confirmed by aspiration or radiography. Once the operator is satisfied that the needle 26 is correctly positioned, the plunger 24 is depressed to push the catheter 1 partially out of the needle 26 causing the branch tubes 5 to open as shown. As mentioned above, the ridges 30 prevent the entire catheter 1 being ejected into the pleural space 51 and also so that the proximal end 15 remains external of the body.

The needle 26 is then withdrawn from the chest and the Y-shape branch tubes 5 retain the catheter 1 in position so that it is not inadvertently withdrawn. The proximal end 15 remains accessible on the exterior side of the chest as shown. Fluid from the pleural space 51 enters the catheter 1 through the holes 7 and travels down the main tube 3 for collection via the proximal end 15 to which is normally attached a bag 30, as shown in FIG. 5. The bag 30 has a connector 31 arranged for detachable connection to the distal end 15 via a corresponding connector 32.

The above-described instrument 20 and catheter 1 allows the procedure to be carried out one-handed, in a relatively straightforward manner, using minimal equipment. The plurality of holes 7 allow continued drainage and prevent or minimise the likelihood of the catheter 1 becoming blocked during drainage.

As shown in FIG. 6, a scalpel 35 (which may be provided as part of an instrument kit, also containing the instrument 20 and the bag 30) may include a blade appropriately marked with a visible indicator 37 which indicates the depth of cut appropriate to the procedure. The size of blade depends on the size of the drain to be used.

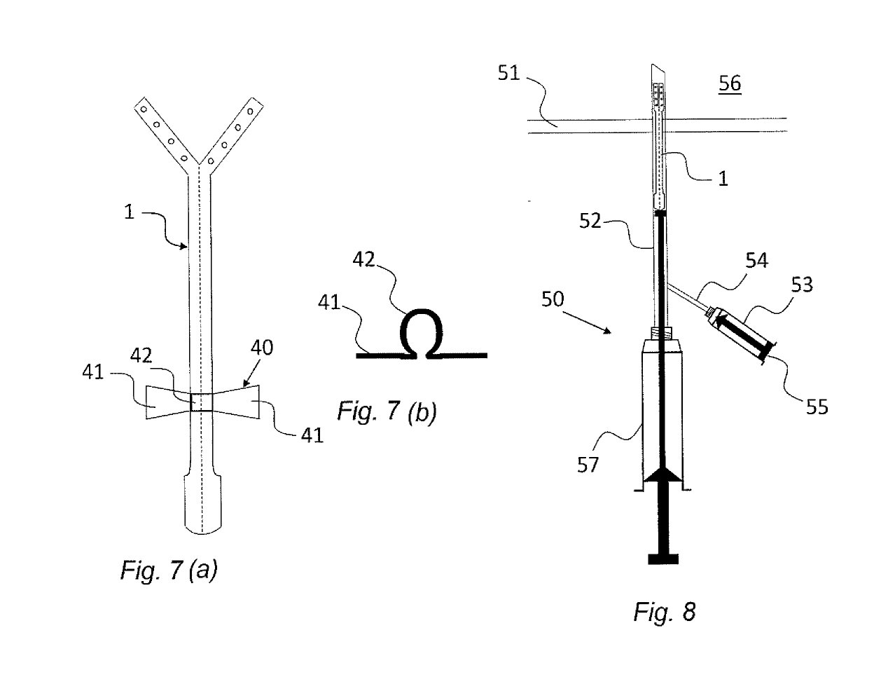

As shown in FIGS. 7a and 7b, a wing-shaped flap 40 may be provided for securing the catheter 1 to the outside of the patient's body after insertion. The flap 40 comprises two wing portions 41 either side of a central arcuate portion 42 which is shaped so as to locate over the main tube 1. The wing portions 41 can be secured using dressing tape or the like, and avoids the need for suturing the catheter 1 to the patient. This may be provided as part of the above-mentioned instrument kit.

The radiopaque marker 17 further allows for identification of the catheter 1 on an X-ray or ultrasound scan, which is useful for positioning.

Referring now to FIG. 8, a second example instrument 50 is shown. The instrument 50 comprises a syringe assembly and the catheter 1 previously described. In the Figure, reference numeral 51 indicates the chest wall, and reference numeral 56 the pleural cavity.

The syringe assembly is similar to that described in relation to FIG. 3, having a needle 52 within which is the preinstalled catheter 1, as well as a syringe barrel 57 within which is located the plunger.

Additionally, there is provided a secondary syringe 53 which connects to the needle 52 by means of a side channel or tube 54.

In use, the chest wall 51 is first cut with a scalpel and the needle 52 inserted into the pleural cavity 56. Confirmation of correct placement is performed by aspirating fluid or air via withdrawal of plunger 55 of the secondary syringe 53. When confirmed, the catheter 1 is inserted as before, i.e. by pushing the plunger of the main syringe so that the distal end of said catheter enters the cavity 56 at which time the branch tubes 5 spring open to the state indicated in FIG. 1. Ridges, as before, limit travel of the plunger so that the proximal end 15 remains within the needle bore, external to the chest wall 51. The instrument 50 is then removed in the same way, leaving the catheter 1 in place with the proximal end 15 exposed externally of the patient's body for draining, securing and collecting in a bag as before.

The second example instrument 50 can be used in other procedures.

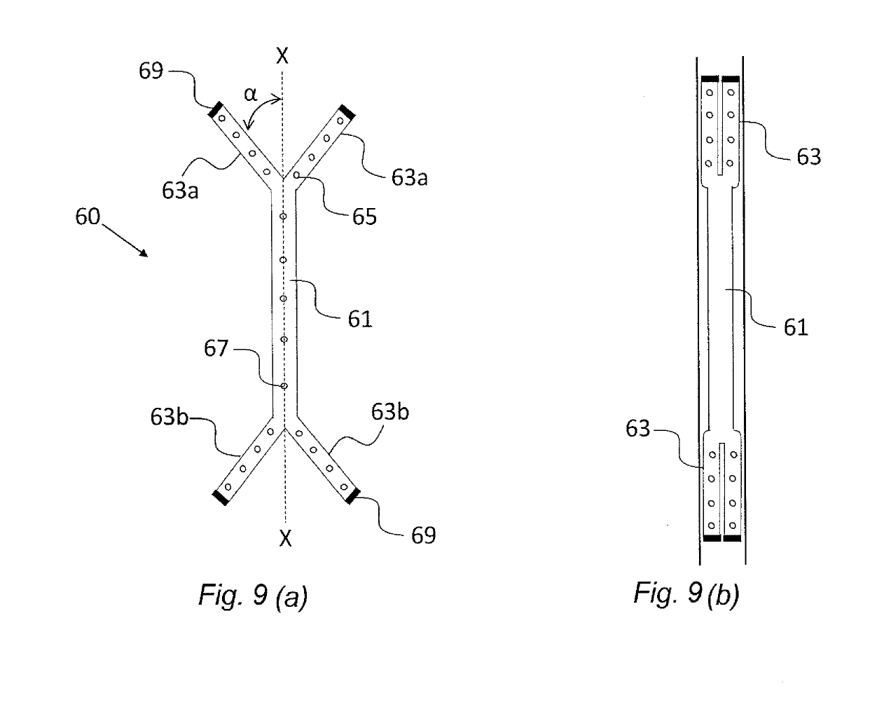

Referring now to FIG. 9a, a second embodiment catheter 60 is shown. The catheter 60 comprises a main, elongate tube 61 extending along lengthwise axis X-X. First and second branch tubes 63a, 63b are joined to each end of the main tube 3 such that the internal passages of the tubes are in communication. Those branch tubes indicated 63a are designated distal branch tubes and those indicated 63b are proximal branch tubes.

There are effectively two Y-shaped portions when unrestricted, or in effect, an elongated X-shape. The branch tubes 63a, 63b each extend away from axis X-X in opposite directions at an angle α which in this case is approximately 40 degrees. As for the first embodiment, the walls of the branch tubes 63a, 63b have a plurality of holes 65 passing from the exterior to the interior passage, the holes extending in a line along the longitudinal extent of each branch tube. In some embodiments, holes may be provided on the opposite (rear) side of the branch tubes 63a, 63b also. In the shown example, each branch tube 63a, 63b has four holes, but different numbers can be used. Further, holes may be provided along the main tube 61 as shown, possibly on both sides.

In some embodiments, a radiopaque marker 69 is provided on the catheter 1. In this case, a marker 69 is provided at or near the ends of each branch tube 63a, 63b for easy identification using an X-ray or ultrasound scan. A fewer or greater number of markers can be used in some embodiments. The entire, or a substantial part of the branch tubes 63a, 63b may carry a marker in some embodiments.

FIG. 9b shows the catheter 60 when located within a needle or trocar passage or bore. It will be appreciated that, when removed from the needle or trocar, the “memory” of the plastics material causes the branch tubes 63a, 63b to return, or spring back, to the initial shape shown in FIG. 9a. As with the first embodiment, the purpose of this is primarily to help self-secure and retain the catheter within a body cavity.

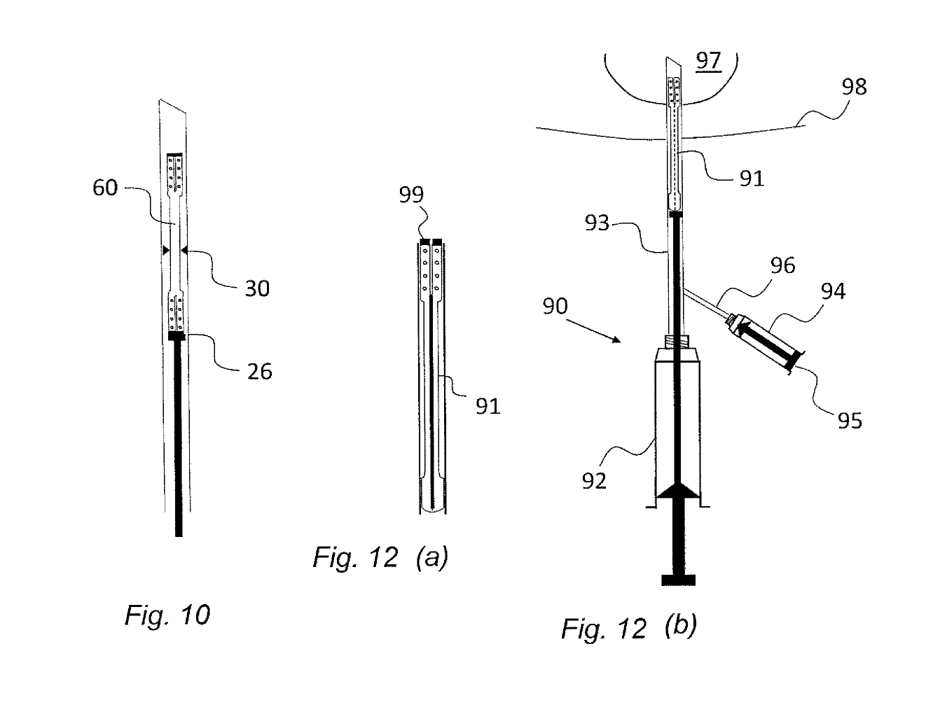

The catheter 60 can be provided as part of a medical instrument comprising a syringe system. In other words, the catheter 60 can be pre-installed in any of the FIG. 3 or 9 instruments in place of the first embodiment catheter 1. FIG. 10 shows in close-up the location of the catheter 60 within the FIG. 3 needle, for example. The ridges 30 or a similar limiting mechanism may be provided for the same purpose.

As before, the instrument can be provided as part of a kit, comprising the instrument with the installed catheter 60, and one or more of a scalpel, and/or instructions. As will be explained, insertion can be achieved using a one-handed insertion action.

The tubes may have a diameter of approximately 3-4 mm (just more than 1 French {Fr) where IFR=3 mm}. The holes may be 1 mm in diameter with the holes placed 2 mm apart.

The tube with a Y configuration at one end will have variable sizes depending upon the size and weight of the subject/patient they are used in.

A number of example procedures using the catheter 60 will now be described.

In one example, the catheter 60 provides a vesico-amniotic shunt, for the drainage of foetal urine. As is known, foetal bladders may have an abnormality in which there is a blockage at the exit which leads to progressive collection of urine. The resulting backpressure can damage the kidneys of the developing foetus. An improved outcome is achieved by bypassing the blockage using a catheter.

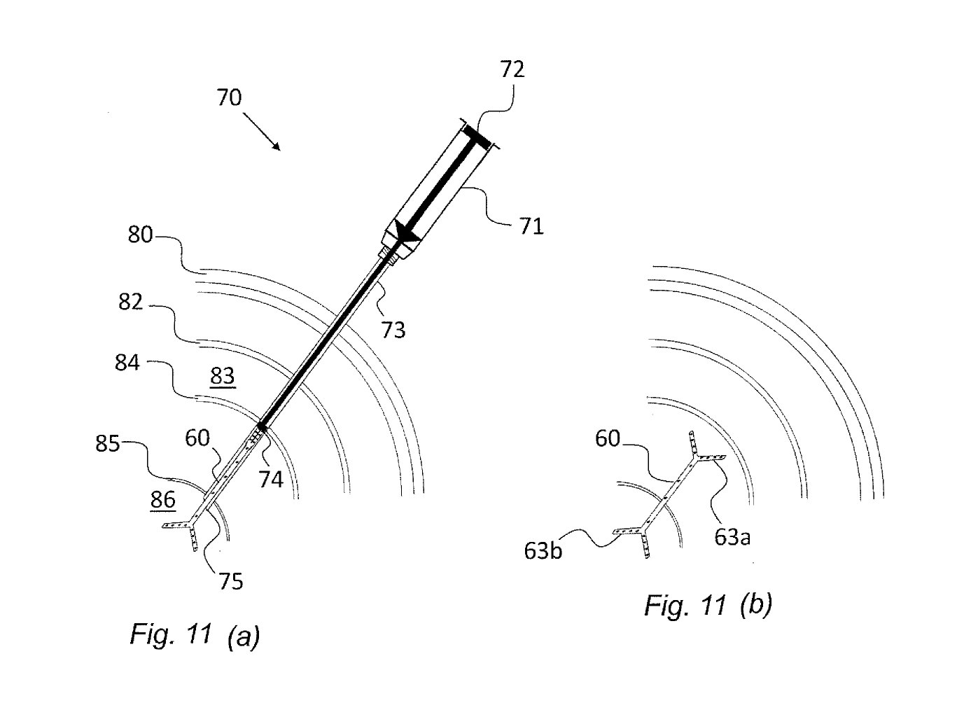

Referring to FIG. 11a, an example instrument 70 is shown in relation to a part-sectional view of a pregnant female abdomen. The abdomen comprises the maternal abdominal wall (MAW) 80, the uterus 82, amniotic fluid (AF) 83, the foetal abdominal wall (FAW) 84, the foetal bladder wall (FBW) 85 and 15 the foetal bladder (FB) 86.

The instrument 70 comprises a syringe barrel 71 housing a plunger 72, a needle (or trocar) 73, and a plunger tip 74 within the needle bore. The shunt 60 is initially pre-installed entirely within the bore, as previously indicated in FIG. 10.

The method of inserting the shunt 60 comprises making an initial incision using a scalpel on the MAW 80, and then passing the needle/trocar 73 through the shown sections until the tip 75 enters the FB 86. This is verified through radiology, e.g. using X-rays or ultrasound, whereby the radio opaque marker(s) 69 indicate the position of shunt ends.

The plunger 72 is then depressed into the barrel 71 so as to eject part of the shunt 60 into the FB 86, as shown in FIG. 11a. The ridges 30 limit movement of the plunger 72 or plunger tip 74 from ejecting the entire shunt 60 from the trocar 72 and leaves the proximal branch tubes 63b on the opposite side of the FBW 85. The branch tubes 63a at the distal end move or return outwardly to the Y-position shown in FIG. 9a, which is verified by radiology using the markers 69. When verified, the instrument 70 is gradually withdrawn, leaving the shunt 60 retained within the FB 86 at one end. As the instrument 70 is removed further, the shunt 60 is fully withdrawn from the trocar 72 and the proximal end branch tubes 63b move outwardly to the Y-position.

FIG. 11b shows the shunt 60 in place following complete withdrawal of the instrument 70. The length of the shunt 60 is such that the proximal branch tubes 63b open within the amniotic sac, this being the region between the FAW 84 and the FBW 85. The installed shunt 60 can be installed in a simple, single-handed operation and results in draining foetal urine from the FB 86 into the amniotic sac. The shunt 60 is self-retaining by virtue of the two sets of branch tubes 63a, 63b which prevents displacement into the bladder.

The instrument 70 with pre-installed shunt 60 can be provided pre-packed in a single size, together with a suitable scalpel blade which can be appropriately marked for the purpose of the procedure.

In a further example, a catheter 91 is provided for use as a subrapubic bladder drainage catheter. After bladder surgery, or in order to bypass a blockage in the bladder, a catheter is used to drain urine. After surgery, this is needed to allow the bladder to heal without distention by urine.

Referring to FIG. 12a, the catheter 91 is similar is shape and form to that shown in FIGS. 1 and 2, having the Y-shaped branch tubes at one end. Radio-opaque markings 99 at provided at or near the distal ends of the branch tubes for radiographic location.

Referring to FIG. 12b, there is shown an instrument 90 for use in the procedure, which comprises a syringe apparatus 92 similar to that shown in FIG. 8 which includes a needle 93 (or trocar) with a side tube 96 connected to a secondary syringe 94 and plunger 95. The catheter 91 is pre-installed within the bore of the needle 93.

Reference numeral 98 indicates the abdominal wall of a patient, and reference numeral 97 the urinary bladder.

In use, an incision is first made in the abdominal wall 98. The needle 93 is introduced as before and confirmation of its tip being within the bladder 97 can be confirmed by aspiration of urine using the secondary syringe 94 and/or radiolocation of the markings 99. The distal end of the catheter 91 is then ejected as before, by depressing the plunger of the syringe apparatus, and the open form of the Y-shaped branch tubes can be confirmed by radiolocation. The instrument 90 is then withdrawn backwards, and the catheter 91 remains in place due to the Y-shaped branch tubes and urine is drained through the proximal end of the catheter, e.g. into a bag.

In all above cases, the medical instruments can be provided as a kit as mentioned in relation to some embodiments, together with one or more associated apparatus.

In all above cases, the catheter can comprise more than two branch tubes in some embodiments. Whilst the branch tubes are shown extending off the ends of the main tube, they may in some embodiments extend off at different locations along the main tube, e.g, near to but not at the end(s). Although the term needle has been used throughout, it will be appreciated that the term applies to any form of hollow introducer element. The resilience of the catheter which causes it to return to its original, unrestrained, shape is by virtue of it being formed in this shape using a suitable plastics material, such as a polymer with elastic properties. In some embodiments, the use of a smart memory material may be considered.

It will be appreciated that the above described embodiments are purely illustrative and are not limiting on the scope of the invention. Other variations and modifications will be apparent to persons skilled in the art upon reading the present application.

Moreover, the disclosure of the present application should be understood to include any novel features or any novel combination of features either explicitly or implicitly disclosed herein or any generalization thereof and during the prosecution of the present application or of any application derived therefrom, new claims may be formulated to cover any such features and/or combination of such features.

We claim:

1. A medical catheter, comprising:
an elongate main tube, the tube having an outer wall which surrounds an internal passage;
first and second branch tubes, each having an outer wall connected to the outer wall of the main tube, which outer wall surrounds an internal passage which is in communication with that of the main tube, wherein the branch tubes extend outwards from the main tube in different respective directions and have one or more apertures passing through their outer wall to their internal passage to permit fluid to pass through the branch tubes and into the main tube in use, and wherein the branch tubes are arranged so as to be resiliently movable, generally towards each other, to permit location of the catheter within the bore of a needle or needle-like apparatus and which return to their original outwards state when removed.

2. The catheter of claim 1, wherein at least part of the main tube extends along a central axis and in which the branch tubes extend outwardly from said central axis on opposite sides to provide an overall Y-shape or similar.

3. The catheter of claim 1, wherein the branch tubes extend from one end of the main tube.

4. The catheter of claim 1, wherein each branch tube comprises a plurality of apertures passing through the side wall.

5. The catheter of claim 4, wherein the apertures are distributed along the branch tube, between the connection with the main tube and a free end of the branch tube.

6. The catheter of claim 1, wherein first and second branch arms are connected at or near one end of the main tube, and at or near the opposite end are provided third and fourth branch arms which extend away from the main tube in different respective directions and have one or more apertures passing through their outer wall to their internal passage to permit fluid to pass through the branch tubes and into the main tube in use, and wherein the third and fourth branch tubes are arranged so as to be resiliently movable, generally towards each other.

7. The catheter of claim 6, wherein one or more apertures are further provided in the wall of the main tube.

8. The catheter of claim 1, further comprising a radiopaque marker.

9. The catheter of claim 8, wherein the radiopaque marker extends along the main tube.

10. The catheter of claim 9, wherein one or more radiopaque markers are provided on respective branch tube(s).

11. The catheter of claim 10, wherein the one or more radiopaque markers is provided on or near the free end of each branch tube.

12. A medical catheter comprising a main tube having first and second ends, and at least two, shorter, branch tubes which extend outwardly from at, or near, the first end of the main tube on opposite sides to form an approximate Y-shape, the branch tubes each having one or more apertures formed through their side wall so as to provide a fluid passage from the branch tube into the main tube, the branch tubes being connected to the main tube in a flexible and resilient manner such that they can be moved together and located within a needle or needle-like structure and which are biased so that they return to their original outwards position when removed therefrom.

13. The catheter of claim 12, wherein a plurality of further such branch tubes are provided at the, or near, the second end of the main tube so that the catheter forms an approximate X shape.

14. An apparatus for locating a catheter in a medical procedure, the apparatus comprising: a barrel having a first end and a second end and a sidewall defining an interior chamber; a needle or needle-like member having first and second ends and a longitudinal passage extending between said ends, the first end of the needle being connected in use to the second end of the barrel, and the second end of the needle being a free-end, the longitudinal passage being arranged in use to house a catheter according to any preceding claim; a plunger locatable within the interior chamber of the barrel for reciprocal movement therein between a non-depressed position and a depressed position and connected to a plunger rod which passes through the barrel second end and part-way along the passage of the needle so as to cause ejection of only part of the catheter from the needle free-end as the plunger is moved towards the depressed position in use.

15. The apparatus according to claim 14, further comprising a catheter pre-installed within the needle, the catheter comprising an elongate main tube, the tube having an outer wall which surrounds an internal passage;
first and second branch tubes, each having an outer wall connected to the outer wall of the main tube, which outer wall surrounds an internal passage which is in communication with that of the main tube, wherein the branch tubes extend outwards from the main tube in different respective directions and have one or more apertures passing through their outer wall to their internal passage to permit fluid to pass through the branch tubes and into the main tube in use, and wherein the branch tubes are arranged so as to be resiliently movable, generally towards each other, to permit location of the catheter within the bore of a needle or needle-like apparatus and which return to their original outwards state when removed.

16. The apparatus according to claim 14, wherein the length of the plunger rod is arranged to cause ejection of part of the catheter sufficient to allow the branch arms to return to the outwards state.

17. The apparatus according to claim 14, further comprising means provided within the needle passage or the barrel for limiting the travel of the plunger rod to cause ejection of part of the catheter to allow the branch arms to return to the outwards state.

18. The apparatus according to claim 17, wherein the limiting means comprises at least one indentation within the needle passage.

19. The apparatus according to claim 14, further comprising a secondary syringe connected to the needle by means of a branch tube for use in aspiration.

20. A medical kit, comprising an apparatus according to claim 14, and one or more of a fluid-collecting bag for connection to the free end of the main tube of a catheter, a scalpel and a retaining member for locating over the catheter for securing it to a patient.

21. The kit of claim 20, wherein the scalpel comprises a blade which is marked so as to indicate visually a depth of cut required for a particular medical procedure.

22. The kit of claim 20, wherein the retaining member comprises a central arcuate portion arranged to locate around part of the catheter wall, and first and second flaps located on opposite sides of the arcuate portion.
